(19) United States
(12) Patent Application Publication
(10) Pub. No.: US 2009/0275970 A1
(43) Pub. Date: Nov. 5, 2009

(54) Surgical access needle device and method of use

(75) Inventor: Carla Leibowitz, San Carlos, CA (US)

Correspondence Address: KILPATRICK STOCKTON LLP - 55461, 1001 WEST FOURTH STREET, WINSTON-SALEM, NC, 27101, US

(73) Assignee: Kyphon SARL, 2000 Neuchatel (CH)

(21) Appl. No.: 12/112,746

(22) Filed: Apr. 30, 2008

(51) Int. Cl. A61B 17/34

(52) U.S. Cl. 606/185

(57) ABSTRACT

A surgical access needle device, systems and kits comprising the surgical access needle device, and methods of using the surgical access needle device can comprise a first elongate member having a blunt distal end, a second elongate member having a sharp distal end, and an actuator mechanism. The actuator mechanism can be operably attached to the first elongate member or second elongate member to adjust the positional relationship between the first elongate member or second elongate, thus exposing a distal end of the first elongate member or the second elongate member. Variations of the devices, systems, kits, and methods are useful for providing and creating a surgical access passage for performing minimally invasive surgery or accessing internal organs or tissues.

### FIELD OF THE INVENTION

The following description relates to a surgical access needle device, systems and kits comprising a surgical access needle device, and methods for using a surgical access needle device.

### BACKGROUND OF THE INVENTION

Conventional surgical procedures for pathologies and/or trauma located deep within the body can cause significant trauma to intervening tissues. Open surgical procedures often require a long incision, extensive muscle stripping, prolonged retraction of tissues, denervation, and devascularization of tissue in order to access a surgical site. Most of these surgeries require several hours of recovery room time and several weeks of post-operative recovery time due to the use of general anesthesia and the destruction of tissue during the surgical procedure. In some cases, these invasive procedures lead to permanent scarring and pain.

Minimally invasive alternatives, such as endoscopic techniques, reduce pain, post-operative recovery time, and the destruction of healthy tissue. In minimally invasive surgery, the site of pathology is accessed through portals rather than through a significant incision, thus preserving the integrity of intervening tissues. These minimally invasive techniques also often require only local anesthesia. The avoidance of general anesthesia can reduce post-operative recovery time and the risk of complications.

Minimally invasive surgical techniques are particularly desirable for spinal and neurosurgical applications because of the need for access to locations deep within the body and the danger of damage to vital intervening tissues. For example, a common open procedure for disc herniation, laminectomy followed by discectomy, requires stripping or dissection of the major muscles of the back to expose the spine. In a posterior approach, tissue including spinal nerves and blood vessels around the dural sac, ligaments, and muscle must be retracted to clear a pathway from the skin to the disc. These procedures normally take at least one to two hours to perform under general anesthesia and require post-operative recovery periods of at least several weeks. In addition to the long recovery time, the destruction of tissue is a major disadvantage of open spinal procedures. As a result, many patients may be reluctant to seek surgery as a solution to pain caused by spinal conditions.

In order to reduce the post-operative recovery time and pain associated with spinal and other procedures, micro-surgical techniques have been developed. For example, in micro-surgical discectomies, the disc can be accessed by creating a pathway from the surface of the patient's back to the disc through a percutaneous puncture or small incision. Small diameter micro-surgical instruments may be passed through the puncture access or small incision and between two vertebrae and into the disc. The intervening tissues are disrupted less because the incision and the exterior-to-interior pathway are smaller. Although these micro-surgical procedures are less invasive, they may still involve some of the same risk of complications associated with open procedures, such as injury to tissue related to penetration with a sharp-tipped access device.

Minimally invasive surgical techniques allow a surgical procedure to be performed on a patient's body through a relatively small puncture access or incision in the body and with a limited amount of body tissue disruption. Minimally invasive surgery typically utilizes a tubular structure known as a cannula which is inserted into a puncture access or small incision in the body. The cannula holds the puncture access or incision open and serves as a conduit extending between the exterior of the body and the local area inside the body where the surgery is to be performed.

To gain initial access to a surgical site, conventional approaches and techniques have been developed, including, for example, inserting a small insertion cannula having a sharp tip, for example a trocar cannula, to penetrate tissue to a surgical site. The sharp tip is often needed to penetrate tough tissue, such as fascia or the annulus of a disc. Upon reaching the surgical site, a guide wire can then be inserted into the insertion cannula, and the insertion cannula can then be removed. When utilizing a sharp tip to penetrate to the surgical site, a risk of causing nerve damage exists. Some of the tissue between the skin and the surgical site does not require a sharp tip to penetrate and/or navigate the tissue; thus the patient is exposed to a risk of nerve damage and other trauma when only a sharp tipped access instrument is utilized.

Another conventional technique includes utilizing a Jamshidi device to create an initial percutaneous route to the surgical site. The Jamshidi device can have several stylets with different tips that can be inserted and removed from the device. However, in order to exchange a tip in a Jamshidi device, the operator must remove one stylet from the device and insert a separate stylet having a different tip into the device. The exchange can be time consuming and may create some risk that the Jamshidi insertion device is moved during the stylet exchange.

Thus, there is a need for devices and methods adapted to create access to a surgical site that reduce the risk of tissue trauma and that increase the efficiency of a surgeon. There is a need for devices and methods that provide for such advantages in percutaneous, minimally invasive surgery useful in a variety of applications and approaches.

### SUMMARY

Described herein are embodiments of a surgical access needle device, systems and kits comprising a surgical access needle device, and methods for using a surgical access needle device that may be useful for creating a surgical access passage for performing minimally invasive surgery or for accessing an internal organ or tissue in a human or animal.

In one embodiment, a surgical access needle device comprises a first elongate member comprising a first distal end having a blunt tip, a second elongate member comprising a second distal end having a sharp tip, and an actuator mechanism. The position of the second elongate member can be coaxial with and slidable relative to the first elongate member. In some embodiments, the actuator mechanism can adjust a positional relationship between the first elongate member and the second elongate member. The actuator mechanism can be operably attached to the first elongate member or the second elongate member. In some embodiments, the actuator mechanism can be configured to adjust the positional relationship between the sharp tip of the second elongate member and the blunt tip of the first elongate member. Some embodiments of the surgical access needle device can allow an operator to switch between the type of tip extended at the distal end of the access needle device within a patient's body. The access needle device can provide an operator with quick access to different styles of tips when providing or creating access to a surgical site or other internal region, so as to efficiently minimize the risk of tissue damage or other trauma to the patient during such process.

Some embodiments can include a system and/or kit comprising a surgical access needle device as described herein. The system and/or kit may further comprise additional components, for example, a surgical access cannula and/or surgical instruments. Yet other embodiments include methods for providing access to a surgical site. Such an embodiment can utilize a surgical access needle device comprising a first elongate member having a blunt tip distal end, a second elongate member having a sharp tip distal end, and an actuator mechanism. In some embodiments, the methods can include exerting and releasing a force upon the actuator mechanism to extend and retract the distal end of the first elongate member and/or second elongate member. In some embodiments where the first elongate member is a guide wire, the method can further include removing the second elongate member having a sharp tip and the actuator mechanism. The guide wire can be used to thread a delivery cannula to the surgical site for passage of surgical instruments in minimally invasive surgical procedures.

Features of a surgical access needle device, systems, kits, and/or methods of using an access needle device may be accomplished singularly, or in combination, in one or more of the embodiments. As will be realized by those of skill in the art, many different embodiments of an access needle device, systems, kits, and/or methods of using an access needle device are possible. Additional uses, advantages, and features of the access needle device, systems, kits, and/or methods of using an access needle device are set forth in the illustrative embodiments discussed in the detailed description herein and will become more apparent to those skilled in the art upon examination of the following.

### BRIEF DESCRIPTION OF DRAWINGS

FIG. 1 is a perspective view of one embodiment of a surgical access needle device showing a first elongate member, a second elongate member, and an actuator mechanism.

FIG. 2A is a enlarged perspective view of one embodiment depicting a section of the surgical access needle device shown in FIG. 1.

FIG. 2B is a perspective view of one embodiment depicting the section of the access needle device shown in FIG. 2A, illustrating the actuator mechanism with a force exerted upon the actuator mechanism.

FIG. 3A is an elevational view of an embodiment depicting the distal end of the access needle device showing a blunt tip extended beyond a sharp tip.

FIG. 3B is an elevational view of an embodiment depicting the distal end of the access needle device showing a sharp tip extended beyond a blunt tip.

FIG. 4A is an elevational view of one embodiment depicting an unassembled collet gripper.

FIG. 4B is an elevational view of one embodiment depicting an assembled collet gripper.

FIG. 5A is a schematic, cross sectional view of one embodiment depicting a collet gripper in an un-tightened position.

FIG. 5B is a schematic, cross sectional view of one embodiment depicting a collet gripper in a tightened position.

FIG. 6A is a side elevational view of one embodiment depicting one variation of a surgical access needle device showing a first elongate member, a second elongate member, and an actuator mechanism.

FIG. 6B is a side elevational view of one embodiment depicting the access needle device shown in FIG. 6A, after activation of the actuator mechanism.

FIG. 7A is a side elevational view of an embodiment depicting an actuator mechanism.

FIG. 7B is a side elevational view of one embodiment depicting the actuator mechanism in FIG. 7A, after a force has been exerted upon the actuator mechanism.

FIG. 8 is a side elevational view of one embodiment depicting the distal end of the access needle device showing the sharp tip in dashed lines within the lumen of the blunt tip.

FIG. 9 is a side elevational, cross-sectional view of one embodiment depicting the distal end of the access needle device.

FIG. 10 is a flow chart of one embodiment illustrating steps for using an access needle device.

### DETAILED DESCRIPTION

Described herein are embodiments of a surgical access needle device, systems and kits comprising a surgical access needle device, and methods of using an access needle device that can be useful for performing minimally invasive surgery or for accessing an internal organ or tissue.

As used in this specification and the appended claims, the singular forms “a,” “an,” and “the” include plural referents unless the context clearly dictates otherwise. Thus, for example, the term “a projection” is intended to mean a single projection or a combination of projections. As used in this specification and the appended claims, “proximal” is defined as nearer to a point of reference such as an origin, a point of attachment, or the midline of the body. As used in this specification and the appended claims, “distal” is defined as farther from a point of reference, such as an origin, a point of attachment, or the midline of the body. Thus, the words “proximal” and “distal” refer to direction nearer to and farther from, respectively, an operator (for example, surgeon, physician, nurse, technician, etc.) who inserts a medical device into a patient, with the tip-end (i.e., distal end) of the device inserted inside the patient's body. For example, the end of a medical device inserted inside the patient's body is the distal end of the medical device, while the end of the medical device outside the patient's body is the proximal end of the medical device.

Some embodiments described herein include a surgical access needle device comprising a first elongate member comprising a first distal end having a blunt tip, a second elongate member comprising a second distal end having a sharp tip, and an actuator mechanism. The position of the second elongate member can be coaxial with first elongate member. In some embodiments, the position of the second elongate member can be slidable relative to the first elongate member. In some embodiments, the actuator mechanism can adjust a positional relationship between the first elongate member and the second elongate member. In some embodiments, the actuator mechanism can be operably attached to the first elongate member or second elongate member. The actuator mechanism can be configured to adjust the positional relationship between the sharp tip of the second elongate member and the blunt tip of the first elongate member. Some embodiments of the surgical access needle device can allow an operator to switch between the type of tip extended at the distal end of the access needle device within a patient's body. In some embodiments, the actuator mechanism provides a mechanism such that an operator may choose whether she desires a blunt or sharp tip in situ (e.g. after the distal end of the access needle device has been positioned in the patient) while also allowing the switching of tips in situ in the patient's body with the application of minimal force.

Other embodiments described herein include a surgical access needle system and/or a surgical access needle device kit. Such a system and/or kit can include embodiments of the access needle device as described herein. For example, the surgical access needle device kit can include the access needle device and a delivery cannula. Yet other embodiments can include a method of using an access needle device to provide access to a surgical site. Such a method can comprise utilizing the surgical access needle device, system, and/or kit as described herein. For example, one such method can include accessing a surgical site, comprising percutaneously inserting toward the surgical site a surgical access needle device as described herein; extending the sharp tip of the second elongate member beyond the blunt tip of the first elongate member; selectively penetrating a first tissue with the sharp tip of the first elongate member; retracting the sharp tip of the second elongate member so that the blunt tip of the first elongate member extends beyond the sharp tip of the second elongate member; and selectively penetrating a second tissue with the blunt tip of the second elongate member.

In conventional surgical procedures, for example in back surgery in the area of a disc, a surgeon or other operator can switch between a blunt stylet and sharp stylet four or more times while accessing the surgical site. Surgeons often rely upon sharp tip access needles in order to pierce and penetrate harder, or tougher, tissue, such as fascia or the annulus of a disc. However, between such tougher tissue is softer tissue such as fat or muscle that a sharp tip needle is not required in order to pierce, penetrate, and/or separate. Risks can be present when utilizing a sharp tipped stylet during the entire procedure as numerous nerve endings and other delicate tissue are present around surgical sites and the path to the surgical site. Inadvertent contact of the sharp tip with a nerve may damage the nerve and/or cause pain to the patient. An operator may not have the ability to rely upon tactile feedback when penetrating tissue with a sharp tip stylet, because often, once contact is made between a sharp tipped stylet and nerve, the damage may occur immediately. To avoid this problem, some surgeons remove the access device and/or switch the tips between blunt and sharp tips depending on which layer of tissue is being penetrated. Each time the stylet is switched using conventional methods, the stylet is removed from the patient in order to the change the tip or to insert a stylet with a different tip. Removing and/or changing stylets can increase procedure time and may possibly dislocate the positioning of the access device.

Embodiments of a surgical access needle device described herein can address the risks and efficiencies of problems associated with conventional methods. In some embodiments, the access needle device can allow an operator to utilize and switch between both a sharp and blunt tip in situ. In some embodiments, the switch or change between a sharp tip and a blunt tip can be quick and efficient, thereby minimizing some risks associated with minimally invasive surgical procedures.

In some embodiments, a surgical access needle device can comprise a first elongate member, second elongate member, and actuator mechanism. The first elongate member can comprise a elongated cylindrical member, such as a cannula, having a proximal end and a distal end. In some embodiments, the distal end of the first elongate member can comprise a blunt tip. In some embodiments, the first elongate member can comprise a guide wire.

The second elongate member can comprise an elongated cylindrical member, such as a cannula having a proximal end and a distal end. In some embodiments, the distal end of the second elongate member can comprise a sharp tip. The first elongate member and the second elongate member can be positioned to have substantially the same center axis, that is, the first and second elongate members can be axially aligned or coaxial. In some embodiments, the first elongate member can be positioned in the lumen of the second elongate member. In other embodiments, the second elongate member can be positioned in the lumen of the first elongate member. Embodiments of the surgical access needle device can be comprised of different sized diameter devices and different sized first and second elongate members.

For example, the first elongate member and second elongate member can have different diameters or cross-sectional dimensions. In some embodiments, the second elongate member can have a larger diameter than the first elongate member. In such embodiments, the second elongate member can be positioned such that an inner surface of the second elongate member can be juxtaposed to an outer surface of the first elongate member. In other embodiments, the first elongate member can have a larger diameter than the second elongate member. In such embodiments, an inner surface of the first elongate member can be juxtaposed to an outer surface of the second elongate member.

The diameters of the first elongate member and second elongate member can have a difference that is great enough in order to allow a sliding clearance between the two members. When positioned concentrically, the member with the larger diameter can be repositioned along the longitudinal axis with minimal frictional interference by the outer surface of the smaller-diameter member.

The first elongate member and second elongate member can comprise material(s) that provide a balance of rigidity and flexibility to facilitate delivery and manipulation of the access needle device to the surgical site, as well as to facilitate delivery and manipulation of additional surgical tools that may be utilized in connection with a minimal invasive surgical procedure, for example delivery cannulas and expandable members. Such materials can include, for example, vinyl, nylon, polyethylenes, ionomer, polyurethane, and polyethylene tetraphthalate (PET), stainless steel, Kevlar™ material, PEBAX™ material, nickel-titanium alloys (Nitinol™ material), and other metal alloys.

Some embodiments of an access needle device can also comprise an actuator mechanism. In some embodiments, the actuator mechanism can be operably connected to the second elongate member. The actuator mechanism may be activated to reposition the second elongate member. For example, in one embodiment, the actuator mechanism can be operably attached to the second elongate member such that application of a force to the actuator mechanism can reposition the second elongate member relative to the first elongate member. As a further illustration, application of force to the actuator mechanism can result in the repositioning of the distal end of the second elongate member as compared to the distal end of the first elongate member. In other embodiments, the actuator mechanism can be operably connected to the first elongate member having a blunt tipped distal end.

For example, prior to applying a force to the actuator mechanism, the blunt distal end of the first elongate member can extend beyond the sharp distal end of the second elongate member. Upon the application of a force to the actuator mechanism, the sharp distal end of the second elongate member can be repositioned such that the sharp distal end of the second elongate member extends beyond the blunt distal end of the first elongate member.

In some embodiments, the actuator mechanism can be activated to reposition the second elongate member repeatedly. In some embodiments, a constant application of force may be required to reposition the first elongate member and second elongate member relative to each other such that the sharp tip of the respective elongate member extends beyond the blunt tip of the other elongate member. Upon the release of force, the first elongate member and second elongate member can return to their initial relative positions so that the blunt tip of the respective elongate member extends beyond the sharp tip of the other elongate member. In some embodiments, upon the application of force to the actuator mechanism, the actuator mechanism may be locked into the position where the sharp tip of the second elongate member extends beyond the blunt tip of the first elongate member.

In some embodiments, the actuator mechanism comprises a button operably attached to a spring, for example a coil spring. Upon an application of force to the button, the first elongate member or second elongate member attached to the button structure can be repositioned. For example, the position of the second elongate member can be shifted such that the distal end of the second elongate member extends beyond the distal end of the first elongate member. Upon the application of force to the button, the spring can be compressed. In some embodiments, upon release of the force applied to the button, the spring decompresses and returns to its initial deactivated state.

As a result of the decompression of the spring, the operably attached second elongate member can be repositioned. In some embodiments, the operably attached second elongate member can return substantially to its original position (i.e., the position prior to the application of force). In some embodiments, upon the decompression of the spring, the distal end of the second elongate member can retract above the position of the distal end of the first elongate member so that the tip of the first elongate member extends beyond the tip of the second elongate member. In some embodiments, the spring can be biased such that the blunt distal end of the first elongate member is continuously exposed until a force is applied to the spring to move the sharp tip beyond the distal end of the blunt tip. The biasing capability, or spring rating, of the spring can be sufficient to retract the sharp tip completely above the blunt tip when activating pressure is released and to maintain the sharp tip above, or more proximal than, the blunt tip.

In some embodiments, the button may be positioned in an axial direction relative to the elongate members. The button can have an arm that extends axially that can be attached to an elongate member such that the attached elongate member can be repositioned upon the repositioning of the button. For example, as the button moves longitudinally, from proximal to distal, the second elongate member, being operable attached to the arm, can move longitudinally.

In some embodiments, the spring can encircle the elongated member such that the spring is coaxial with the elongated member. The proximal end of the spring can be in contact with the distal surface of the arm of the button such that the spring is operably attached to the button. For example, the application of a downward longitudinal force on the button can result in downward longitudinal force exerted upon the spring such that the spring can be compressed. Upon release of the force, the spring can decompress with a force sufficient to move the button toward the proximal end of the access needle device.

For example, in connection with minimally invasive surgical procedures, a force can be applied to the button to expose the sharp distal end of the second elongate member to pierce through hard or tough tissue. Upon piercing and penetration of the tough tissue, the operator may no longer prefer a sharp distal end to be exposed and instead prefer a blunt distal end to be the leading end. The force applied to the button can be released such that the distal end of the second elongate member retracts above the blunt distal end of the first elongate member. With the blunt distal end of the first elongate member being exposed, the operator may more safely penetrate softer tissue to the surgical site.

In some embodiments, the actuator mechanism can be positively activated with a continuing pressure, e.g. by an operator's thumb or finger. In this way, when the pressure on the actuator mechanism is released and the actuator mechanism is deactivated, the actuator mechanism and attached second elongate member (and sharp tip) are automatically returned to their initial, unactivated position so that the sharp tip is retracted above the blunt tip.

In other embodiments, the actuator mechanism can comprise a screw. The screw can be configured such that upon applying a force to the screw to rotate the screw, the operably attached first elongate member or second elongate member can be repositioned. For example, in some embodiments, the screw can be operably attached to second elongate member such that upon rotating the screw clockwise or counter-clockwise, depending on the thread orientation, the second elongate member can be repositioned relative to the position of the first elongate member such that the position of the distal end of the second elongate member can be adjusted upon the rotation of the screw. In some embodiments, the screw can be attached to the outer surface of the elongate member such that the rotation of the screw corresponds to the rotation of the elongate member. The handle of the surgical access needle device can comprise a matingly threaded cavity in which the screw passes. The screw being attached to the elongate member can be rotated within the threaded cavity to change the position of the screw and the attached elongate member.

For example, the screw can be rotated to expose the sharp distal end of the second elongate member to pierce through tough tissue. Upon piercing and penetration of the tough tissue, the operator may no longer prefer a sharp distal end and instead prefer a blunt distal end to be exposed. The screw can be rotated to retract the distal end of the second elongate member such that the blunt distal end of the first elongate member can be exposed.

In other embodiments, the actuator mechanism can comprise a lever and a spring, for example, a coil spring. Upon application of force to the lever, the first elongate member or second elongate member attached to the lever can be repositioned. For example, the position of the second elongate member can be shifted such that the distal end of the second elongate member extends beyond the distal end of the first elongate member. Also upon the application of force to the lever, the spring can be compressed. In some embodiments, as the spring is compressed, the distal end of the second elongate member can be extended. In some embodiments, upon release of the force applied to the lever, the spring decompresses and returns to its initial, deactivated state.

In some embodiments, the lever may be positioned in a substantially axial direction relative to the elongate members. The lever can have an arm that extends axially that can be attached to an elongate member such that the attached elongate member can be repositioned upon the repositioning of the lever. For example, as a downward force is applied to the lever causing the lever to rotate around a pivot point, the second elongate member, being operably attached to the arm, can move in a longitudinal (proximal-to-distal) direction.

In some embodiments, the spring can encircle the elongated member such that the spring is coaxial with the elongated member. The proximal end of the spring can be in contact with the distal surface of the arm of the lever such that the spring is operably attached to the lever. For example, the application of a downward force on the lever, causing rotation around the pivot point, can result in downward longitudinal force exerted upon the spring such that the spring can be compressed. Upon release of the force, the spring can decompress with a force sufficient to re-rotate the lever around the pivot point toward the initial, deactivated position of the lever.

As a result of the decompression of the spring, the operably attached first elongate member or second elongate member can be repositioned. In some embodiments, the operably attached first elongate member or second elongate member can return substantially to its original position (i.e., the position prior to the original application of force). In some embodiments, upon the decompression of the spring, the distal end of the second elongate member can retract above the position of the distal end of the first elongate member such that the tip of the first elongate member extends beyond the tip of the second elongate member.

In some embodiments, the surgical access needle device can comprise a handle. The handle can provide a structure for the operator to hold the device. In some embodiments, the handle can provide a structure to house the actuator mechanism. In some embodiments, the handle can comprise a structure to which a collet gripper (as described below) can attach. In some embodiments, the features of the collet gripper can be included in the handle. The handle may comprise a central hollow cavity configured to allow a first elongate member or second elongate member to pass directly through the handle.

In some embodiments, the surgical access needle device further comprises a collet gripper. In some embodiments, the collet gripper can be a torque-providing device. The torque-providing device can comprise a collet with a collar and tightening device. In some embodiments, the torque-providing device can comprise a Tuohy-Borst adapter. In some embodiments, the torque-providing device can be a guide wire torquer. The collet gripper can attach to the first elongate member such that upon attachment, the collet gripper can provide a stabilizing mechanism. For example, the collet gripper can be attached to the handle such that upon repositioning of the second elongate member, the first elongate member is held stationary relative to the handle. In some embodiments, the collet gripper can be configured to release the first elongate member. For example, upon providing access to a surgical site, the operator can release the collet gripper from the first elongate member and remove the second elongate member, handle, and collet gripper, thus leaving the first elongate member in the desired position.

Certain embodiments of a surgical access needle device can include an indicator mechanism to indicate when the surgical access needle device is in desired position(s) or which distal end is extended. For example, components of the access needle device, such as the distal ends of the first elongate member and the second elongate member can include radiopaque indicators, and the procedure can be visualized under fluoroscopy.

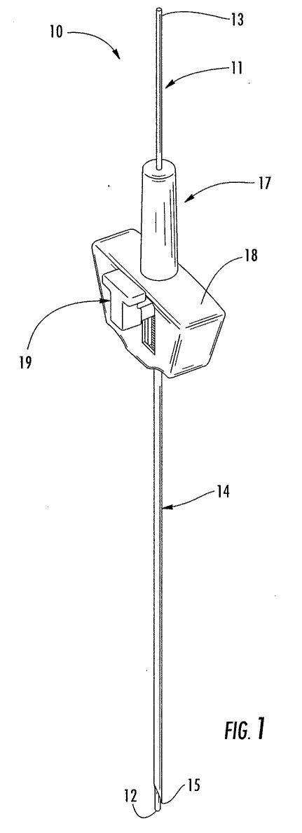

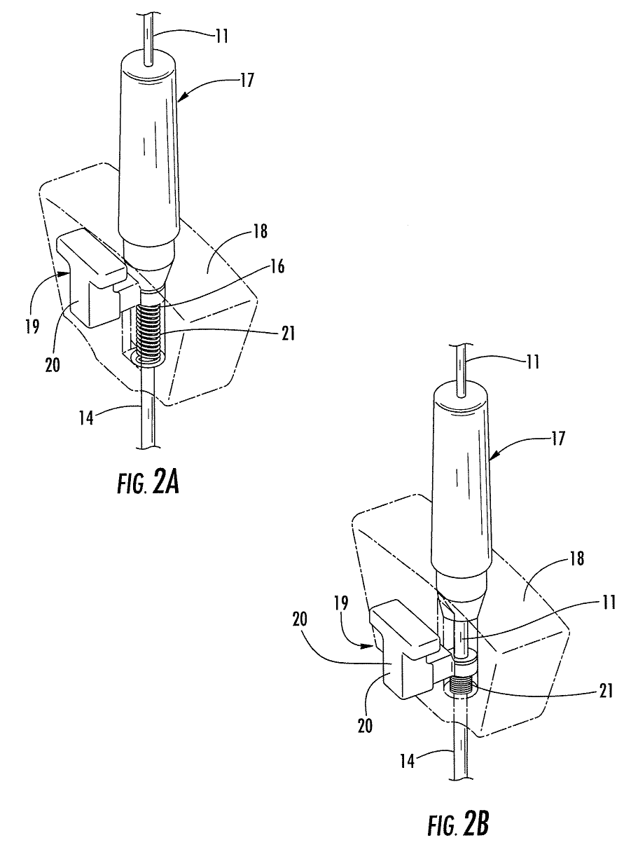

Referring now to the figures, in the embodiment shown in FIGS. 1-5, a surgical access needle device 10 may comprise a first elongate member 11 having a proximal end 13 and a distal end 12 and a second elongate member 14 having a proximal end (not visible in FIG. 1, see FIG. 2 for illustration) and a distal end 15. The access needle device 10 can additionally comprise an actuator mechanism 19. In the embodiment shown in FIG. 1, the actuator mechanism 19 comprises a button. The handle 18 can provide a structure in which the operator can use to control the access needle device 10. Collet gripper 17 can be positioned on first elongate member 11 in proximity to the handle 18. In the embodiment shown in FIG. 1, actuator mechanism 19 comprises a spring-loaded button coupled to second elongate member 14. However, actuator mechanism 19 can comprise any mechanism for changing the relative positions of first elongate member 11 and second elongate member 14, including a lever(s), a trigger(s), a knob(s), scissor-type handles, a geared mechanism, a hydraulic system, an electromagnetic actuator, or any combination, among other mechanisms.

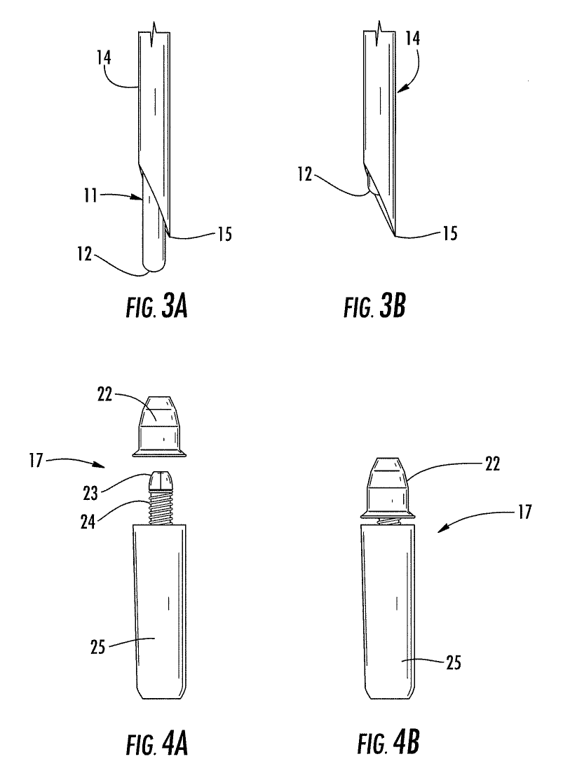

In FIG. 1, the distal end 12 of the first elongate member 11 has a blunt tip. The distal end 15 of the second elongate member 14 has a sharp tip. The first elongate member 11 is positioned within the lumen of second elongate member 14. In the embodiment depicted in FIG. 1, the blunt distal end 12 of the first elongate member 11 extends beyond the sharp distal end 15 of the second elongate member 14. FIG. 3A provides an enlarged view of the blunt distal end 12 extending beyond the sharp distal end 15. In some embodiments, the initial, or original, resting positions of the distal ends of the first elongate member and second elongate member can be the orientation depicted in FIG. 3A, that is, with the blunt tip of the first elongate member extending beyond the sharp tip of the second elongate member.

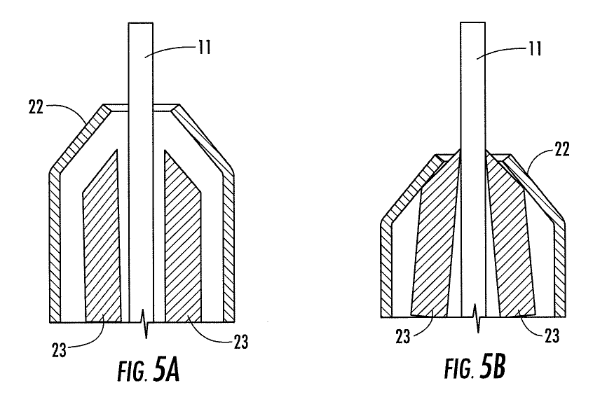

FIG. 2A provides an enlarged view of the access needle device of FIG. 1 in section depicting the actuator mechanism 19. The handle 18 has been depicted as transparent to illustrate the actuator mechanism 19 comprising a button 20 and spring 21. The actuator mechanism 19 can be operably attached to the second elongate member 14. In the embodiment shown, the actuator mechanism is operably attached to the second elongate member 14 in the proximal region 16 of the second elongate member 14. The collet gripper 17 can be positioned on the first elongate member 11 to provide a device to hold the first elongate member 11 relatively stationary upon activation of the actuator mechanism 19 (the collet gripper being described below in connection with FIGS. 4 and 5). The collet gripper 17 can be affixed to the handle 18 to provide a stationary structure relative to the second elongate member. Thus, upon activation of the actuator mechanism 19 and the resulting movement of the second elongate member 14, the first elongate member 11 having the collet gripper 17 affixed, remains in a relative stationary position with respect to the second elongate member.

FIG. 2B provides an enlarged view of the access needle device of FIGS. 1 and 2A after the actuator mechanism has been activated. FIG. 2B depicts the access device upon the application of force to the actuator mechanism 19. In the embodiment depicted in FIG. 2B, the button 20 comprises a T-shaped bar that extends outward from the longitudinal axis of the elongate member. The button 20 can comprise an arm extending internally into the handle 18. The distal surface of the arm of the button 20 contacts the spring 21. The arm of the button 20 can be attached to the proximal end 16 of second elongate member 14.

In FIG. 2B, the button 20 has been repositioned upon the application of a downward force. As the button 20 is pushed downward, the spring 21 is compressed and the proximal end 16 of the second elongate member 14 is repositioned (resulting movement of the second elongate member in the distal direction). When the force applied to the button 19 is released, the spring 21 can decompress and return the second elongate member 14 to the position as depicted by the second elongate member 14 in FIG. 2A.

As a force is applied to the button 20, the first elongate member 11 remains relatively stationary with respect to the second elongate member 14. The collet gripper 17 and handle 18 provide an anchoring mechanism such that the first elongate member 11 is stationary. The position of the second elongate member 14 is altered upon the application and removal of downward, or proximal-to-distal, force upon the button 20. The second elongate member 14 slides along the outer surface of the first elongate member 11 such that the distal end 15 of the second elongate member can be exposed or retracted.

Referring to FIGS. 3A and 3B, the relative positions of the blunt tip of the distal end 12 of the first elongate member 11 and of the sharp tip of the distal end 15 of the second elongate member 14 can be viewed. The positions of the distal ends 12, 15 in FIG. 3A correspond to the exemplary embodiment shown in FIG. 2A, while the positions of the distal ends 12, 15 in FIG. 3B correspond to the exemplary embodiment shown in FIG. 2B.

In FIG. 3A, the blunt distal end 12 of the first elongate member is exposed and in position to be utilized as the lead end when creating a surgical access pathway. FIG. 3A represents an embodiment when the actuator mechanism is not activated, i.e. when no force is being applied to the actuator mechanism. In FIG. 3B, the sharp distal end 15 of the second elongate member is exposed and in a position to be utilized as the lead end for creating surgical access pathway. Upon application of a force to the actuator mechanism (not shown), the second elongate member 12 slides along the first elongate member 11 to be repositioned such that the sharp distal end 15 is exposed. In some embodiments, the sharp distal end 15 can be extended beyond the blunt distal end 12 by a distance that allows operation of the sharp end. For example, in some embodiments the sharp distal end 15 extends beyond the distal end 12 by at least about 1 millimeter. In some embodiments, the distal end 15 can extend beyond distal end 12 by at least about 2 millimeters. In other embodiments, the distal end 15 can extend beyond distal end 12 by at least about 6 millimeters.

FIGS. 4A and 4B depict an embodiment of a collet gripper 17 as found in FIGS. 1 and 2. FIG. 4A depicts an exploded view of an exemplary unassembled collet gripper. FIG. 4B depicts an exemplary assembled collet gripper as may be found in embodiments of a surgical access needle device. Cap 22 can be threaded upon threads 24 located in the base 25 of the collet gripper 17. Cap 22 has a first end and a second end, where the first end has a larger cross section than the second end. The first end of cap 22 is closest to the base 25. As cap 22 is threaded onto the threads 24 in base 25, the collar 23 can be covered. The collet gripper 17 can comprise a channel through which the first elongate member 11 (not shown) can be extended. Collar 23 comprises an adjustable diameter ring structure that encircles the first elongate member 11 (not shown). The operation of the collet gripper tightening function is described below in connection with FIGS. 5A and 5B.

FIGS. 5A and 5B depict cross-sectional views of the cap region of the collet gripper 17 in both the un-tightened position and the tightened position. In FIG. 5A, cap 22 is in an un-tightened position. First elongate member 11 is positioned in a lumen extending through the collet gripper 17. The collar 23 has a minimum diameter that allows the free movement of first elongate member 11 along the length of the collet gripper lumen.

In FIG. 5B, the cap 22 is tightened upon the collar 23. Upon the tightening of cap 22 by threading cap 22 upon the threads 24, the minimum diameter of the collar 23 decreases and pinches upon the first elongate member 11. The tightened configuration shown in FIG. 5B anchors the first elongate member 11 from moving freely along the length of the collar gripper lumen. Thus, the movement of the first elongate member 11 in the proximal or distal direction (without the movement of the entire device 10) is eliminated.

Referring back to FIGS. 1 and 2, the collet gripper 17 in the assembled, tightened configuration can prevent the first elongate member 11 from moving relative to the handle 18 and/or second elongate member 14. The collet gripper 17, being attached to the first elongate member 11, physically prevents the first elongate member 11 from changing positions upon the application of a force to the actuator mechanism 19. In some embodiments, the collet gripper 17 can be configured to release the first elongate member 11. For example, upon providing access to a surgical site, the operator can release the collet gripper 17 from the first elongate member 11 and remove the second elongate member 14, handle 18, and collet gripper 17, thus leaving the first elongate member 11 in the desired position.

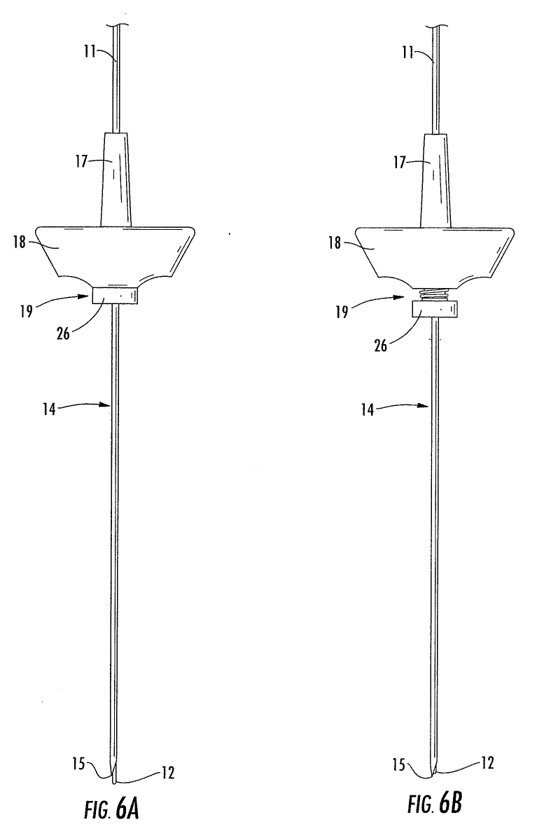

Referring now to the embodiment shown in FIGS. 6A and 6B, the surgical access needle device 10 may comprise a first elongate member 11 having a proximal end 13 and a distal end 12; a second elongate member 14 having a proximal end 16 and a distal end 15; and an actuator mechanism 19 comprising a screw 26. The handle 18 can provide a structure that the operator can use to control the access needle device 10. Collet gripper 17 can be positioned on first elongate member 11 in proximity to the handle 18 near the proximal end 13 of the first elongate member 22.

Similar to the embodiment shown in FIG. 1, the embodiment shown in FIGS. 6A and 6B comprises a distal end 12 having a blunt tip. The distal end 15 of the second elongate member has a sharp tip. The first elongate member 11 can be positioned within the lumen of the second elongate member 14. In the positions of the components in the embodiment depicted in FIG. 6A, the blunt distal end 12 of the first elongate member 11 extends beyond the sharp distal end 15 of the second elongate member 14. FIG. 3A provides a closer view of the blunt tip of the distal end 12 extending beyond the sharp tip of the distal end 15. The screw 26 can be operably attached to the second elongate member 14 in proximity to the proximal end 16 of the second elongate member 14. As previously described, the collet gripper 17 can be positioned on the first elongate member 11 to hold the first elongate member 11 in a stationary position relative to the handle 18.

In FIG. 6A, the screw 26 is positioned in proximity to the handle 18. When the screw 26 is positioned in proximity to the handle 18, the distal end 15 of second elongate member 14 is retracted above the distal end 12 of the first elongate member 11 so that the blunt tip of the distal end 12 of the first elongate member 11 extends distally beyond the sharp tip 15 of the second elongate member 14. To change the relative position of the distal ends 12, 15 the operator rotates the screw 26 to increase the distance between the screw head and the handle 18. As the screw 26 is repositioned, downwardly or distally, relative to the handle 18, the second elongate member 14 is repositioned such that the distal end 15 extends beyond the distal end 12.

Referring to FIGS. 3A and 3B, the relative positions of the distal end 12 of the first elongate member 11 and the distal end 15 of the second elongate member 14 can be viewed. The positions of the distal ends in FIG. 3A correspond to the exemplary embodiment shown in FIG. 6A, while the positions of the distal ends in FIG. 3B correspond to the exemplary embodiment shown in FIG. 6B. To retract the distal end 15 above the distal end 12, the screw 26 can be rotated in the opposite direction such that the screw 26 and the second elongate member 14 move in the proximal direction.

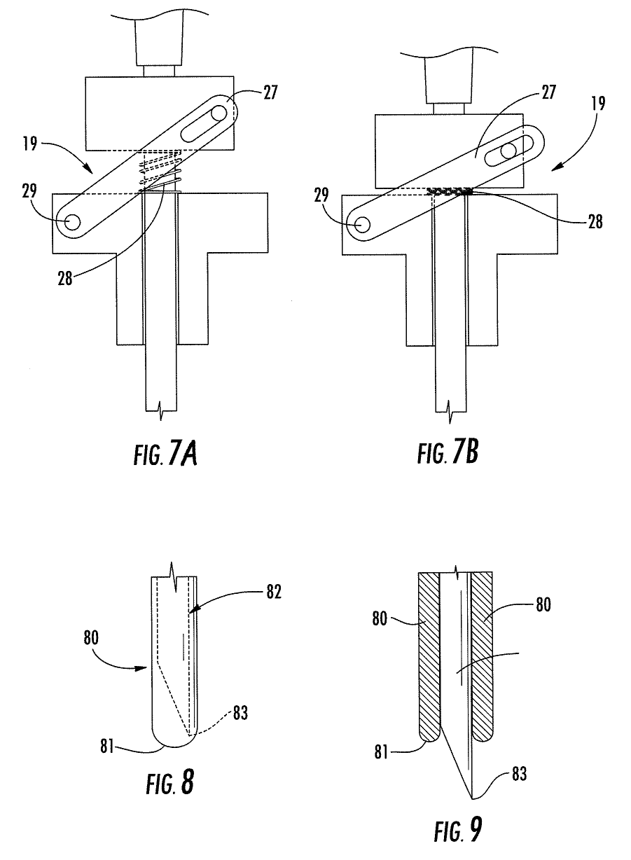

In the embodiments shown in FIGS. 7A and 7B, the actuator mechanism 19 comprises a lever 27 operably attached to a spring 28. The lever 27 can additionally be operably attached to the second elongate member 14 (not shown). The lever 27 has a pivot point 29 upon which the lever 27 pivots. As similarly described in connection with FIGS. 1 and 2, upon application of a force on the lever 27, the position of the second elongate member 14 can be moved downward, or distally, and the spring 28 can be compressed. When the lever 27 is activated, the distal end 15 of the second elongate member 14 (not shown) is extended beyond the distal end 12 of the first elongate member 11. The positions of the distal ends 12, 15 in FIG. 3A correspond to the exemplary embodiment shown in FIG. 7A, while the positions of the distal ends 12, 15 in FIG. 3B correspond to the exemplary embodiment shown in FIG. 7B.

To retract the distal end 15 of the second elongate member 14 above the distal end 12 of the first elongate member 11, the force applied upon the lever 27 can be removed, the spring 28 can decompress, and the distal end 15 of the second elongate member 14 can retract in the proximal direction. In some embodiments, the lever 27 can be locked into position such that sharp distal end 15 of the second elongate member 14 is extending beyond the distal end 12 of the first elongate member 11.

In the embodiment shown in FIG. 8, the first elongate member 80 comprises a distal end 81 having a blunt end and the second elongate member 82 comprises a distal end 83 having a sharp end. The second elongate member 82 is positioned in the lumen of the first elongate member 80. FIG. 9 depicts a cross sectional view of the distal end 83 of the second elongate member 82 extended beyond the distal end 81 of the first elongate member 80. In such embodiments, the actuator mechanism (not shown) can be operably attached to the first elongate member 80. The actuator mechanism can operate in a manner as described herein in addition to those modifications that would be apparent to one of ordinary skill in the art having read the above description so as to operate the first elongate member 80 inside the lumen of the second elongate member 82.

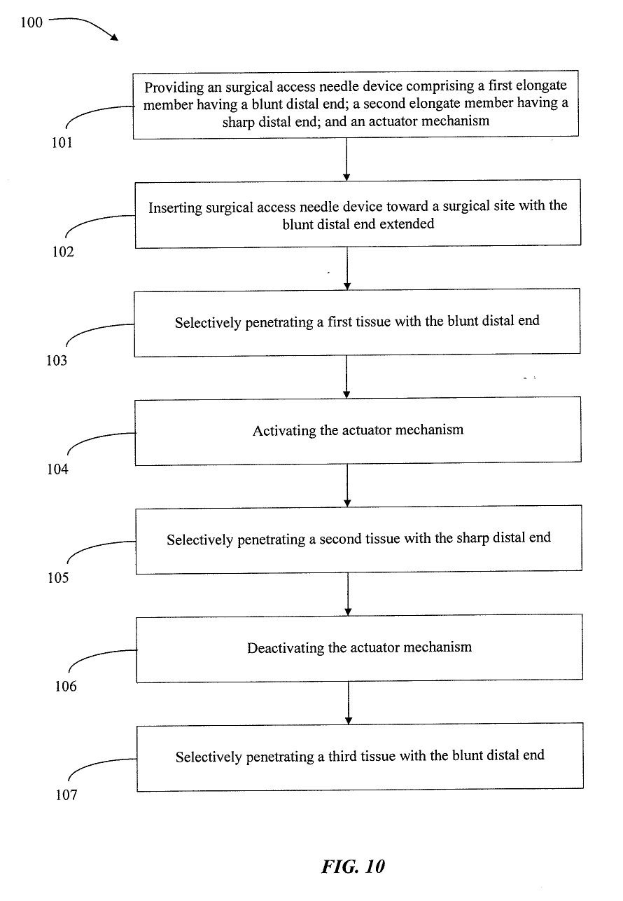

Additional embodiments include methods for using an access needle device to provide access to a surgical site for the surgical or other treatment of a human or animal. FIG. 10 illustrates an embodiment of such a method 100. In such an embodiment, the surgical access needle device 10, as described herein, may be provided (101). The surgical access needle device 10 can be percutaneously inserted toward the surgical site with the blunt distal end of the first elongate member 11 exposed (102). With the blunt distal end exposed, the operator can decrease the risk of tissue damage or other trauma associated with punctures using a sharp tip. The sharp tip can be in a retracted position while the operator penetrates soft tissue.

Upon reaching a tougher tissue in which a blunt tip cannot pierce or penetrate, the operator can apply force to an actuator mechanism to switch from exposure of the blunt distal end to exposure of the sharp distal end without having to remove the device, or any portion of the device, from the patient (104). The sharp distal end of the second elongate member 14 can pierce the tough tissue and penetrate tough tissue as selected by the operator (105). Once the sharp tip is no longer necessary, the operator can remove the force applied to the actuator mechanism, thus retracting the sharp tip (106) so that the blunt tip of the distal end of the first elongate member 11 extends distally beyond the sharp tip of the distal end of the second elongate member 14. With the blunt distal end being extended, the operator can more safely penetrate the tissue with a lower fear of causing tissue damage or other trauma (107). The above described steps 104-107 can be repeated any number of times until the surgical site is accessed.

In embodiments in which the first elongate member 11 comprises a guide wire, an additional step may be performed to provide surgical access to the surgical site. In some embodiments, the second elongate member 14 and the actuator mechanism 19 can be removed from the surgical site. The guide wire can then be used for threading a delivery cannula accurately to the surgical site. When the delivery cannula is in a desired position, the guide wire can be removed.

Embodiments of an access needle device, system, kit, and method of using an access needle device can be utilized for facilitating surgical access in minimally invasive surgical procedures and/or accessing other internal organs or tissues. Some embodiments can be advantageously used in accessing, stabilizing, and fusing of a joint, particularly an intervertebral joint. Some embodiments may be applicable for use with various types of joints (for example, intervertebral, ankle, interdigital, etc.) and in various anatomical regions (for example, spine, arms, legs, etc.) of a human or animal body. In the spinal column, the devices and methods disclosed may be used at all intervertebral joints, including those in the cervical, thoracic, and lumbar region. Some embodiments can be used for a diagnostic procedure, such as a Function Anaesthetic Discography™ (F.A.D.™) Procedure, or a therapeutic procedure, such as procedure providing epidural anesthesia.

Although this description refers to particular embodiments, it should be recognized that these embodiments are merely illustrative of novel and non-obvious principles. Those of ordinary skill in the art will appreciate that a surgical access needle device, system, kit, and methods for using a surgical access needle device may be constructed and implemented in other ways and embodiments. In addition, where methods and steps described above indicate certain events occurring in a particular order, those of ordinary skill in the art having the benefit of this disclosure would recognize the ordering of certain steps may be modified and that such modifications are in accordance with the description herein. Additionally, certain steps may performed concurrently in a parallel process when possible, as well as performed sequentially as described above. Accordingly, the description herein should not be read as limiting such embodiments, as other embodiments also fall within the scope of this disclosure.

What is claimed is:

1. A surgical access needle device, comprising:
a first elongate member comprising a first distal end having a blunt tip;
a second elongate member comprising a second distal end having a sharp tip, the second elongate member coaxial with and slidable relative to the first elongate member; and
an actuator mechanism adapted to adjust a positional relationship between the sharp tip and the blunt tip.

2. The device of claim 1, further comprising the first elongate member positioned in a lumen of the second elongate member.

3. The device of claim 1, further comprising the second elongate member positioned in a lumen of the first elongate member.

4. The device of claim 1, wherein the first elongate member further comprises a guide wire.

5. The device of claim 1, wherein activation of the actuator mechanism extends the sharp tip of the second elongate member beyond the blunt tip of the first elongate member, and deactivation of the actuator mechanism retracts the sharp tip of the second elongate member so that the blunt tip of the first elongate member extends beyond the sharp tip of the second elongate member.

6. The device of claim 5, wherein the actuator mechanism is actuatable by a continuous manual pressure, wherein release of the manual pressure deactivates the actuator mechanism such that the position of the sharp tip of the second elongate member is automatically adjusted so that the blunt tip of the first elongate member extends beyond the sharp tip of the second elongate member.

7. The device of claim 1, wherein the actuator mechanism further comprises a button operably attached to a spring, the button and spring being positioned at a proximal end of the second elongate member and configured such that the button is attached to the spring in an axial direction relative to the elongate members.

8. The device of claim 1, wherein the actuator mechanism further comprises a screw, the screw being configured such that the sharp tip of the second elongate member is repositionable relative to the blunt tip of the first elongate member upon rotation of the screw.

9. The device of claim 1, wherein the actuator mechanism further comprises a lever operably attached to a spring, the lever and spring being positioned at a proximal end of the second elongate member and configured such that the sharp tip of the second elongate member is repositionable relative to the blunt tip of the first elongate member upon activation of the actuator mechanism and deactivation of the actuator mechanism.

10. The device of claim 1, further comprising a collet gripper positionable on the first elongate member and configured to anchor the first elongate member to a handle such that the first elongate member remains stationary relative to the handle upon the activation and deactivation of the actuator mechanism.

11. The device of claim 10, wherein the collet gripper is releasable from the first elongate member such that the first elongate member remains stationary at a surgical site and the handle, second elongate member, and collet gripper are selectively removable from the first elongate member.

12. A method of accessing a surgical site, comprising:
percutaneously inserting toward the surgical site a surgical access needle device comprising a first elongate member comprising a distal end having a blunt tip and a second elongate member comprising a distal end having a sharp tip, the second elongate member coaxial with and slidable relative to the first elongate member;
extending the sharp tip of the second elongate member beyond the blunt tip of the first elongate member;
selectively penetrating a first tissue with the sharp tip of the second elongate member;
retracting the sharp tip of the second elongate member so that the blunt tip of the first elongate member extends beyond the sharp tip of the second elongate member; and
selectively penetrating a second tissue with the blunt tip of the second elongate member.

13. The method of claim 12, the surgical access needle device further comprising an actuator mechanism operably attached to the second elongate member, wherein the extending the sharp tip of the second elongate member further comprises activating the actuator mechanism.

14. The method of claim 13, wherein the retracting the sharp tip of the second elongate member further comprises deactivating the actuator mechanism.

15. The method of claim 14,
wherein activating the actuator mechanism further comprises exerting a force upon the actuator mechanism, and
wherein deactivating the actuator mechanism further comprises releasing the force exerted upon the actuator mechanism.

16. The method of claim 15,
wherein activating the actuator mechanism further comprises exerting the force upon a button operably attached to a spring such that the spring is compressed upon activating the actuator mechanism, and
wherein deactivating the actuator mechanism further comprises releasing the force exerted upon the button such that the spring decompresses upon deactivating the actuator mechanism.

17. The method of claim 16, wherein upon deactivating the actuator mechanism, the spring is biased such that the blunt tip of the first elongate member extends beyond the sharp tip of the second elongate member.

18. The method of claim 12, wherein the surgical site comprises an intervertebral disc.

19. The method of claim 12, wherein the first elongate member comprises a guide wire.

20. The method of claim 12,
wherein the first tissue comprises a tough tissue, and
wherein the second tissue comprises a soft tissue.
